(19) United States
(12) Patent Application Publication
(10) Pub. No.: US 2013/0023947 A1
(43) Pub. Date: Jan. 24, 2013

(54) Implantable Heart Stimulator

(71) Applicant: Andreas Blomqvist, Taby (SE)

(72) Inventor: Andreas Blomqvist, Taby (SE)

(73) Assignee: ST. JUDE MEDICAL AB, Jarfalla (SE)

(21) Appl. No.: 13/514,273

(22) PCT Filed: Dec. 9, 2009
(86) PCT No.: PCT/SE2009/051396

§ 371 Date: Jun. 6, 2012

(51) Int. Cl. A61N 1/365

(52) U.S. Cl. 607/23

(57) ABSTRACT

Implantable heart stimulator comprising a control unit including a memory, a sensing unit, a pulse stimulation unit adapted to generate stimulation pulses separated by a variable predetermined pacing interval (PI), and also a method in a heart stimulator. The heart stimulator is adapted to be connected to one or many heart electrode leads provided with stimulating and sensing electrodes in order to stimulate heart tissue by said stimulation pulses and sense electrical heart events. The heart stimulator comprises a control parameter measurement unit adapted to derive a control parameter value indicative of end-diastolic pressure (EDP). At specified intervals, the control unit is adapted to vary the predetermined pacing interval (PI) according to a predetermined pacing interval (PI) search session scheme, and that control parameter values are determined, by said control parameter measurement unit at the different pacing intervals tested during said PI search session, and in that determined control parameter values and corresponding pacing intervals are stored in said memory. The maximum control parameter value obtained during one PI search session is determined and the corresponding pacing interval, denoted PIₒₚₜ, is identified and used when stimulating the heart resulting in a maximal end-diastolic pressure.

### FIELD OF THE INVENTION

The present invention relates to implantable heart stimulators, such as pacemakers or implantable cardioverter/defibrillators (ICDs), and a method in such stimulators, according to the preambles of the independent claims. In particular the invention relates to techniques for deriving the progression of heart failure within a patient in which a heart stimulator is implanted.

### BACKGROUND OF THE INVENTION

Heart failure is a debilitating disease in which abnormal function of the heart leads in the direction of inadequate blood flow to fulfill the needs of the tissues and organs of the body. Typically, the heart loses propulsive power because the cardiac muscle loses capacity to stretch and contract. Often, the ventricles do not adequately eject or fill with blood between heartbeats and the valves regulating blood flow become leaky, allowing regurgitation or back-flow of blood. The impairment of arterial circulation deprives vital organs of oxygen and nutrients. Fatigue, weakness and the inability to carry out daily tasks may result. Not all heart failure patients suffer debilitating symptoms immediately. Some may live actively for years. Yet, with few exceptions, the disease is relentlessly progressive. As heart failure progresses, it tends to become increasingly difficult to manage. Even the compensatory responses it triggers in the body may themselves eventually complicate the clinical prognosis. For example, when the heart attempts to compensate for reduced cardiac output, it adds muscle causing the ventricles to grow in volume in an attempt to pump more blood with each heartbeat. This places a still higher demand on the heart's oxygen supply. If the oxygen supply falls short of the growing demand, as it often does, further injury to the heart may result. The additional muscle mass may also stiffen the heart walls to hamper rather than assist in providing cardiac output. A particularly severe form of heart failure is congestive heart failure (CHF) wherein the weak pumping of the heart leads to build-up of fluids in the lungs and other organs and tissues. Heart failure is a world wide epidemic and has caught the interest of the cardiology community for the last decade. In screening for HF patients the echo cardiography measure ejection-fraction (EF) has since long been used to evaluate the systolic function of the patient. If a patient shows up with the traditional heart failure symptoms such as fatigue, shortness of breath, excessive fluid retention, etc., and has an EF below 30 or 35% they are most often considered to have heart failure, or more precisely systolic heart failure (SHF). Lately, focus has been directed more and more to a subgroup of patients who shows up presenting signs of HF, namely the ones who have a more or less preserved EF/systolic function. The size of this group is under debate, mostly due to the fact that the definition of this group is somewhat vague, but an estimate of between 30-50% of the patients with HF symptoms is a number most would agree to. These patients have a more or less intact systolic function, but they instead suffer from a diastolic dysfunction and if heart failure symptoms are presented, they are referred to as having diastolic heart failure (DHF), or simply heart failure with diastolic dysfunction.

It is important to point out that diastolic dysfunction is not only present in patients with heart failure symptoms, but in fact, diastolic dysfunction can be present in regular bradycardia patients and/or patients with an implantable cardioverter/defibrillator (ICD) as well. There is a high probability that it will progress into heart failure if undiagnosed but the problem at hand is still the diastolic dysfunction.

EP-1588738 relates to a system and a method for evaluating heart failure based on ventricular end-diastolic volume using an implantable medical device. Values representative of ventricular end-diastolic volume (EDV) are detected and then heart failure, if occurring within the patient, is detected based on the values representative of ventricular EDV. Hence, ventricular EDV is generally used as a proxy or surrogate for ventricular end-diastolic pressure. By using ventricular EDV instead of ventricular end-diastolic pressure, heart failure can be detected and evaluated without requiring sophisticated sensors or complex algorithms. In particular, ventricular EDV can be easily and reliably measured using impedance signals sensed by implanted ventricular pacing/sensing electrodes. If heart failure is detected, then appropriate therapy is automatically delivered by pacer/ICD. Control parameters for CRT therapy are automatically adjusted based on the severity of the heart failure.

EP-1348463 relates to a heart monitoring device that is adapted to derive an impedance value indicative of the impedance between electrode surfaces. The device is in particular used to detect and treat a systolic dysfunction of a heart.

Taken the above into account the object of the present invention is to achieve an improved therapy for heart failure patients, especially those with diastolic dysfunction.

### SUMMARY OF THE INVENTION

The above-mentioned object is achieved by the present invention according to the independent claims.

Preferred embodiments are set forth in the dependent claims.

The present invention is in particular advantageous for patients having an impaired filling phase (diastole). By an initial calibration procedure, preferably performed at implantation, a control parameter indicative of end-diastolic pressure (EDP) is achieved by using e.g. intracardiac (cardiogenic) impedance measurements.

A closed-loop system will then continuously optimize a pacing interval, e.g. the AV-delay, such that EDP is maximized rather then the stroke volume or dP/dt which are more of “systolic parameters” (even though Starlings law will guarantee that some of the additional filling will also lead to an improved ejection). In addition, by optimizing on EDP, mitral valve insufficiency is also accounted for.

The present invention is also advantageous in many other aspects, e.g. with regard to that a therapy is available for patients with diastolic dysfunction and may also easily be included in all device populations (in fact, it would be very advantageous to treat the diastolic dysfunction before it leads to HF) in that the resulting therapy is primarily based upon impedance measurements and IEGM based analysis—no new sensors are required. The inventive stimulator and method uses a simple and intuitive algorithm with sophisticated add-on if desired, and provides a learning system which will only get better and better the more it is used.

### SHORT DESCRIPTION OF THE APPENDED DRAWINGS

FIG. 1 shows a schematic block diagram illustrating the implantable heart stimulator according the present invention.

FIG. 2 shows in a schematic way the relationship between EDP and AV-delay applicable in the present invention, in order to illustrate the connection between these parameters.

FIG. 3 shows a curve illustrating the relationship between optimal pacing interval and heart rate.

FIG. 4 is a flow diagram illustrating the method according to the present invention.

FIG. 5 is a flow diagram illustrating one embodiment of the present invention applied during exercise.

### DETAILED DESCRIPTION OF PREFERRED EMBODIMENTS OF THE INVENTION

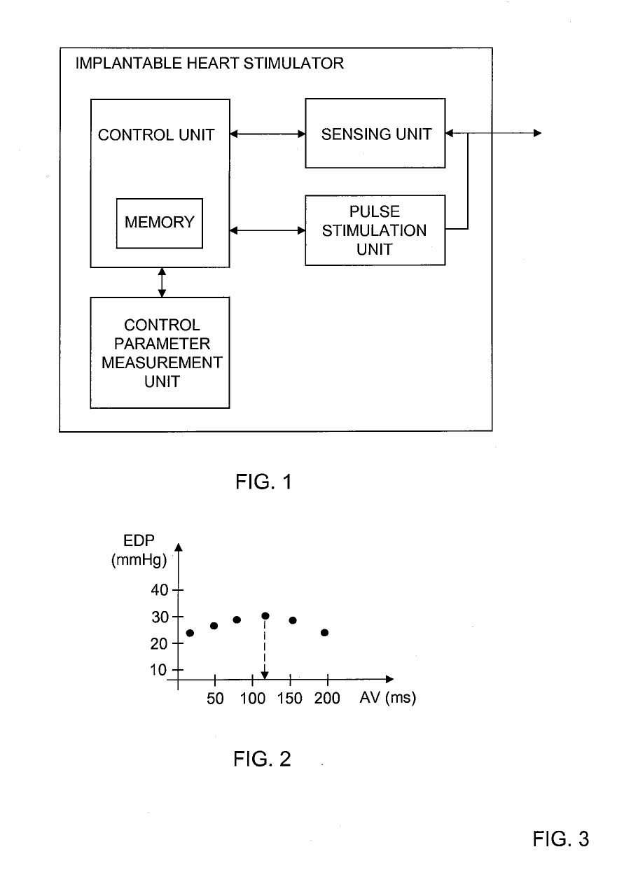

With references to the schematic block diagram in FIG. 1 the implantable heart stimulator according to the present invention will know be described in detail.

The implantable heart stimulator comprises a control unit including a memory, a sensing unit to sense electrical heart events, and a pulse stimulation unit adapted to generate stimulation pulses separated by a variable predetermined pacing interval (PI). The heart stimulator is adapted to be connected to one or many heart electrode leads provided with stimulating and sensing electrodes in order to stimulate heart tissue by the stimulation pulses and to sense electrical heart events. The heart electrode leads are not shown in the figure but indicated via arrows from the sensing and pulse stimulating units.

The electrode leads may be arranged to stimulate the heart in the right ventricle, right atrium, the left atrium and the left ventricle. The leads may be inserted into the respective heart chamber, e.g. for stimulation in the right atrium or ventricle, or adapted to be inserted into the great veins and/or coronary sinus for stimulation of the left atrium and/or ventricle.

Furthermore, the heart stimulator is also provided with a telemetry circuit (not shown) adapted to perform communication, preferably bi-directional communication, to an external programming device. The communicated information may relate to set-up instructions to the stimulator, e.g. with regard to stimulation mode, historic data read out from the memory of the control unit, and software updates, etc.

Many implantable heart stimulators are also provided with an activity sensor for generating an activity signal when the patient is active, e.g. during exercise. The activity sensor may be an accelerometer arranged to sense movements in e.g. three directions.

The heart stimulator, according to the present invention, also comprises a control parameter measurement unit adapted to derive a control parameter value indicative of end-diastolic pressure (EDP).

At specified intervals, preferably being in the range of 6-24 hours, the control unit is adapted to vary the predetermined pacing interval (PI) according to a predetermined pacing interval (PI) search session scheme. Control parameter values are determined, by the control parameter measurement unit at the different pacing intervals tested during the PI search session. The determined control parameter values and corresponding pacing intervals are stored in the memory, along with the current heart rate at the time of this measurement.

The maximum control parameter value obtained during one PI search session is determined and the corresponding pacing interval, denoted PIₒₚₜ, is identified and used when stimulating the heart resulting in a maximal end-diastolic pressure.

The pacing interval is preferably varied in relation to the PIₒₚₜ during the PI search session.

According to a one embodiment the control parameter measurement unit is adapted to be initially calibrated by end-diastolic pressure measurements performed in order to obtain control parameters associating control parameter values to end diastolic pressure values. The control parameter measurement unit is adapted to receive, via telemetry, calibration data as a result of the end-diastolic pressure measurements.

Preferably the end-diastolic pressure measurements are performed by an external blood pressure measurement unit provided with a guide wire, having a pressure sensor at its distal end, adapted to be inserted into a ventricle of the heart, in order to sense end-diastolic pressure (EDP) within at least one of the ventricles of the heart.

As an alternative the end-diastolic pressure measurements may be performed by an external pulsed Doppler ultrasound measurement unit to achieve a non-invasive estimate of left ventricular EDP.

According to a preferred embodiment the control parameter measurement unit is an impedance measurement unit that is adapted to determine cardiogenic impedance (CI) parameters, being the control parameters referred to above. The impedance measurement unit is then used to determine CI parameter values, being the above-mentioned control parameter values, and to store the determined CI parameter values in the memory. The cardiogenic impedance is preferably measured by use of one or many electrodes of the electrode lead(s).

The heart rate (HR), which preferably is obtained from intracardiac electrogram (IEGM) sensed by the sensing unit, is also stored in the memory in relation to stored PI and control parameter values.

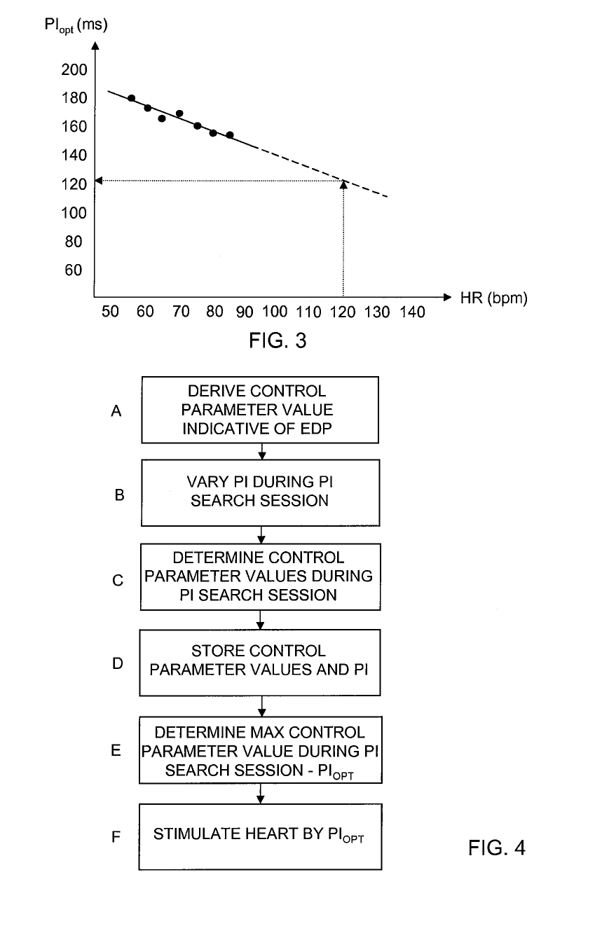

According to an embodiment the control unit is adapted to determine an HR/PIₒₚₜ-relationship based upon related HR- and PIₒₚₜ-values. The determined HR/PIₒₚₜ-relationship is used, by the control unit, to determine an optimal pacing interval for a sensed heart rate. This is illustrated in FIG. 3 where the solid line represents an interpolated linear relationship between the heart rate and the optimal pacing interval PIₒₚₜ and the dashed line an extrapolated linear relationship.

In the figure is illustrated that an identified heart rate of approximately 120 beats per minute corresponds to an optimal pacing interval of 125 ms.

The predetermined pacing interval (PI) is preferably the AV-interval, i.e. the time between a sensed or stimulated atrial event and a stimulated ventricular event.

The predetermined pacing interval may also be the VV-interval, i.e. the time between a sensed or stimulated ventricular event in the right ventricle and a stimulated event in the left ventricle.

Referring to the flow diagram in FIG. 4, the present invention also relates to a method in an implantable heart stimulator comprising:

A) deriving a control parameter value indicative of end-diastolic pressure (EDP);

B) varying, at specified intervals, preferably in the range of 6-24 hours, a predetermined pacing interval (PI) during a PI search session according to a predetermined PI search session scheme;

C) determining control parameter values at the different pacing intervals tested during the PI search session;

D) storing determined control parameter values and corresponding pacing intervals;

E) determining the maximum control parameter value obtained during one PI search session and identifying the corresponding pacing interval, denoted PIₒₚₜ, and

F) stimulating the heart by PIₒₚₜ resulting in a maximal end-diastolic pressure.

In step B, the pacing interval is preferably varied in relation to said PIₒₚₜ during the predetermined PI search session.

An initial calibration is preferably performed by using end-diastolic pressure measurements by sensing end-diastolic pressure (EDP) within at least one of the ventricles of the heart in order to obtain control parameters associating control parameter values to end diastolic pressure values.

The heart rate (HR) is also stored in relation to stored PI and control parameter values.

According to one embodiment of the method the cardiogenic impedance (CI) parameters are determined, being the control parameters, and used to determine CI parameter values, being the control parameter values, and to store the determined CI parameter values.

The pacing interval is the AV-interval or the VV-interval.

Given the present rate at which the pacing interval (in this case the AV-delay) is varied, each such occasion will result in a plot as schematically depicted in FIG. 2. Based on that data, the most appropriate AV-delay is chosen such that it maximizes EDP even though it would potentially be a sub-optimal setting for forward flow (i.e. dP/dtₘₐₓ, SV or aortic VTI)—and that is the core of the therapy for diastolic dysfunction. This guarantees optimal filling for the stiffening myocardium and it will also create an acceptable output level due to the mechanism of Starlings law which basically states that with a higher preload a more powerful ejection follows. FIG. 2 shows actual data from the CICOR study of the resulting true EDP in mmHg when varying the AV-delay. This is the way that the result of an AV-sweep in accordance with the present invention would look, the only difference being that the actual values on the y-axis would be the control parameter value, e.g. the impedance derived EDP estimate, and not the true EDP as in this particular plot. The figure serves to show that there will be a maximum EDP value for a specific AV-delay and that it is the delay that should be chosen (here indicated by the arrow) as the optimal pacing interval denoted PIₒₚₜ.

In the following the present invention will be described in relation to two embodiments where an external measurement device is used in order to obtain end-diastolic pressure values and where cardiogenic impedance parameter values are used as control parameter values. Herein the AV delay is used as the pacing interval (PI).

Initially, for calibration purposes, a control parameter is determined which in this case is performed by creating an impedance based model for end-diastolic pressure (EDP). This is done by using a reference that must be as good as possible. One excellent choice for good EDP measurement is the Radi Medical's PressureWire™ which is arranged in the left ventricle during the implantation procedure of the IMD. The cardiogenic impedance (CI) is then recorded simultaneously as the EDP-recording is being made, and this is to be repeated at approximately five or more AV delays (which preferably is separated as much as possible, i.e. from 200 ms to 30 ms in even intervals). During each AV setting one or multiple impedance vectors will be recorded. The example used in herein is based upon a real case and data collected in the so called CICOR human study. The vectors used will be referred to as V1 and V3 respectively and are defined for V1 as i: RVring-LVring, u: RVtip-LVtip, and for V3 as i: RVring-RVtip, u: RVtip-RVtip (i=current for the injection nodes and u=voltage for the sensing nodes). RVring and LVring are the ring electrode of the right and left ventricular electrode lead, respectively. RVtip and LVtip are the tip electrode of the right and left ventricular electrode lead, respectively.

Note that this is one possible set-up for determining the cardiogenic impedance, but several other alternatives are also plausible within the scope of the present invention as being defined by the appended claims.

Each vector recording during each AV setting will generate one “template” which is an ensemble average waveform, preferably obtained through an approximately 20 seconds long recording of CI. From these waveforms parameters (or features) are extracted. These may be the rate of change during systole (dZ/dt_{syst}), peak-to-peak amplitude, the time of the maximum value relative to the R-wave, etc. As long as these parameters are not strictly a mere representation of rate it is acceptable to include a large number of parameters without risk of over training the model. These parameters are then used to calculate the linear model that best predicts the reference, which in this case was EDP obtained by the pressure measurement device (PressureWire™). This can be done in several ways but one of the most powerful ways to create this model is to use a multivariate analysis toll, for instance SIMCA, to handle the necessary calculations. The entire procedure above may be repeated again for different VV-timings if the patient has a CRT device (although the AV-delay is the parameter which has the largest effect on EDP and also means that this therapy is applicable in most IMD patients, not only those with CRT-devices).

The implantable heart stimulator according to the present invention thus includes a fully automatic device based estimate of the EDP. The calibration process described above, will have to be performed on each individual patient as described above but once in place, no further manual involvement is needed. The managing physician will (after the calibration process) be prompted to activate (or not) the diastolic dysfunction therapy. When activated, the algorithm will on regular specified intervals perform scans of different PI intervals (e.g. five different AV delays) and predict the corresponding maximum EDP using the impedance based model. The specified intervals may be e.g. 6-24 hours, once a week or any other suitable interval, and will be valid for that specific hart rate present at the time of the scan.

In one embodiment the present invention is to apply the heart stimulator and method in connection to exercise as well, as patients with diastolic dysfunction are very sensitive to higher heart rates. This would require some more sophisticated implementation though that will be disclosed below when describing an exercise application, in which the pacing interval is the AV-delay and the control parameter is cardiogenic impedance (CI).

In the following an exercise application of the present invention will be described. The system described above will in general make sure that the patient who suffers from diastolic dysfunction always gets the best possible filling. However, if we assume that the patient is living an active life and is impeded in doing so from his/her disease—of course there will not be enough time for the system to run through a complete AV- (or VV-) sweep and then choose the appropriate setting, the physical exertion may already be over as this takes a couple of minutes and that the entire scan would have to be completed during the higher heart rate achieved during the exercise. It would also be directly inappropriate to start to change the AV delay in an unphysiological or hemodynamically deleterious manner during exercise as that could have dire consequences of the patient's health.

This situation is handled in the following way. The AV-searches at which the system acquires the resulting EDPs will be performed with intrinsic atrial activity (if possible). This means that every time the AV-search is performed the system will also note which rate the patient was in at this particular time. Given some time with this therapy switched on, the system will have gathered EDPs at different heart rates and a trend can be seen, as schematically shown in FIG. 3.

FIG. 3 is a schematic picture illustrating how the algorithm may derive which AV-delay that would result in the highest EDP at a certain heart rate, even though no previous data exist at that heart rate and there is no time for a new calibration process. The dots indicate the results of previous AV-sweeps at different heart rates (in this case around 70 bpm), that is which AV-delay that give rise to the highest ECP. Extrapolating a linear or exponential (to be determined empirically) fit to these points we may now produce a well founded guess of what the optimal AV-delay would be given a certain heart rate.

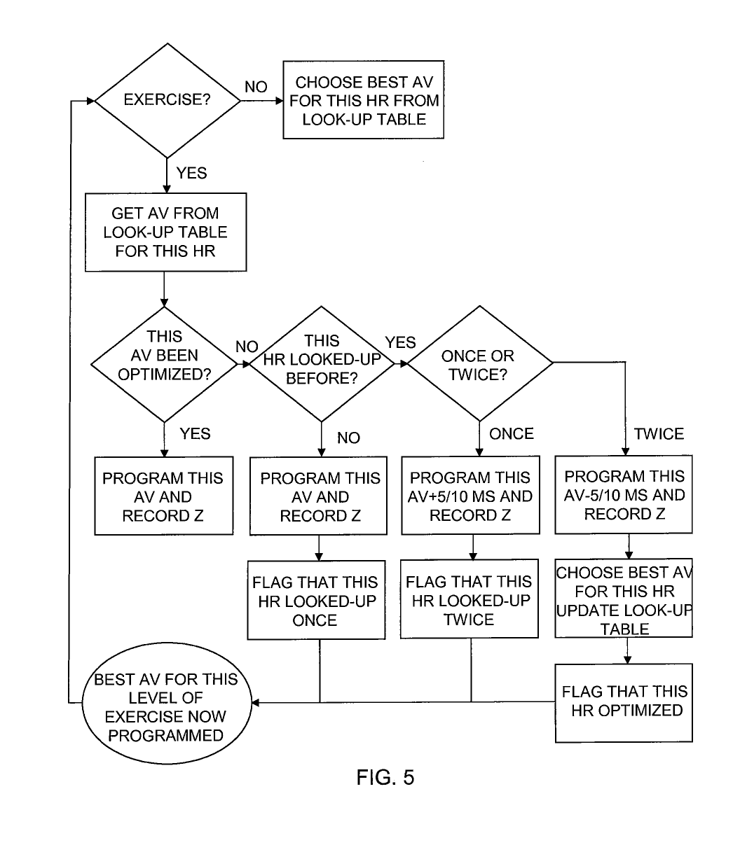

FIG. 5 shows a flow diagram illustrating an embodiment of the present invention to be applied for active patients.

The relationship illustrated in FIG. 3 allows us to extrapolate and approximate what AV- (and potentially VV-) setting that would give the best EDP given a certain heart rate. So once the activity sensor shows that the patient is active, the heart rate is sensed and the algorithm can look into a look-up table and see what the suggested AV-setting is. The look-up table is a tabulated representation of the relationship between the pacing interval, e.g. AV-delay, and the heart rate.

Even though no sweep of different AV-delays will be made during this high-activity period, the EDP (as estimated by impedance) will be calculated at this given heart rate and at the AV-delay suggested by the look-up table. The next time exercise happens the system will check if the look-up table has been used once for this specific HR before. If so, it will use the suggested AV-delay but add an offset of 5-10 ms. The next consecutive time, i.e. the second time, the offset will instead be subtracted from the original guess. In the algorithm illustrated in FIG. 5 the optimal AV-delay is determined, based upon the resulting EDP, based upon three measurements. More advanced parabolic interpolation methods may be used to identify, with a higher resolution, the AV-delay yielding maximum EDP in the vicinity of the three available points.

However and more generally, the optimal pacing interval for a given heart rate may be identified by identifying a local maxima of a curve determined as an approximation of the available measurement EDP data, which is illustrated in FIG. 2, i.e. the approximation of the curve is constantly updated and not limited to only three different AV-delay to identify an optimal AV-delay as in the algorithm shown in FIG. 5.

At all of these instances the EDP is estimated and a learning system is achieved that can be used to fine-tune the look-up table for certain heart rates that are recurring.

The additional information will be added to the database in the memory thus creating a learning system that will predict the appropriate AV-delay even better the next exercise occasion.

The algorithm in FIG. 5 is preferably constantly activated and paused only during the scans described in FIG. 4.

Note that even though the examples above are limited to the use of two specific impedance vectors as they have an empirically proven efficacy, and it is primarily focused on AV-delays as the pacing interval (PI), it has been shown that the present invention easily may be generalized to incorporate more impedance vectors and also VV-delays with the same arithmetic as for the AV-delays. Furthermore, other correlates may be included apart from EDP that also may be used to optimize the diastolic properties, rather than the forward flow properties, and also include those in the therapy for these patients.

The present invention is not limited to the above-described preferred embodiments. Various alternatives, modifications and equivalents may be used. Therefore, the above embodiments should not be taken as limiting the scope of the invention, which is defined by the appending claims.

1-20. (canceled)

21. An implantable heart stimulator comprising:
a control unit comprising a memory, a sensing unit, a pulse stimulation unit adapted to generate stimulation pulses separated by a variable predetermined pacing interval (PI), wherein the heart stimulator is adapted to be connected to at least one lead provided with one or more electrodes adapted to stimulate heart tissue by said stimulation pulses and to sense electrical heart events, wherein the control unit is adapted to vary said predetermined pacing interval (PI) according to a predetermined pacing interval (PI) search session scheme;
a control parameter measurement unit adapted to derive a control parameter value indicative of end-diastolic pressure (EDP) for the various pacing intervals tested during said PI search session, wherein the control parameter measurement unit determines the control parameter value corresponding to a maximal EDP and selects the corresponding pacing interval PIₒₚₜ for stimulating the heart.

22. The implantable heart stimulator according to claim 21, wherein said control parameter measurement unit is adapted to be initially calibrated by end-diastolic pressure measurements performed in order to obtain control parameters associating control parameter values to end diastolic pressure values.

23. The implantable heart stimulator according to claim 22, wherein said control parameter measurement unit is adapted to receive calibration data as a result of said end-diastolic pressure measurements.

24. The implantable heart stimulator according to claim 21, wherein the heart rate (HR), obtained by said sensing unit, is also stored in said memory in relation to stored PI and control parameter values.

25. The implantable heart stimulator according to claim 24, wherein said control unit is adapted to determine an HR/PIₒₚₜ-relationship based upon related HR- and PIₒₚₜ-values.

26. The implantable heart stimulator according to claim 25, wherein the determined HR/PIₒₚₜ-relationship is used, by said control unit, to determine an optimal pacing interval for a sensed heart rate.

27. The implantable heart stimulator according to claim 21, further comprising an impedance measurement unit that is adapted to determine cardiogenic impedance (CI) parameters, being said control parameters, and to store the determined CI parameter values in said memory.

28. The implantable heart stimulator according to claim 21, wherein said predetermined pacing interval is the AV-interval.

29. The implantable heart stimulator according to any of claim 21, wherein said predetermined pacing interval is the VV-interval.

30. The implantable heart stimulator according to claim 21, wherein said specified intervals are in the range of 6-24 hours.

31. The implantable heart stimulator according to claim 21, wherein the pacing interval is varied in relation to said PIₒₚₜ during said predetermined pacing interval (PI) search session.

32. In an implantable heart stimulator, a method comprising:
varying a pacing interval (PI) during a PI search session to test a plurality of PI;
deriving a control parameter value indicative of end-diastolic pressure (EDP) for each PI;
storing determined control parameter values and corresponding pacing intervals;
determining the control parameter value corresponding to a maximal EDP obtained during the PI search session and identifying the corresponding pacing interval, denoted PIₒₚₜ, and
stimulating the heart using PIₒₚₜ.

33. The method of claim 32, wherein initial calibration is performed by using end-diastolic pressure measurements in order to obtain control parameters associating control parameter values to end diastolic pressure values.

34. The method of claim 33, wherein said end-diastolic pressure measurements is performed by sensing end-diastolic pressure (EDP) within at least one of the ventricles of the heart.

35. The method of claim 32, wherein the heart rate (HR) is also stored in relation to stored PI and control parameter values.

36. The method of claim 32, wherein cardiogenic impedance (CI) parameters are determined and used as the control parameter values.

37. The method of claim 32, wherein said predetermined pacing interval is the AV-interval.

38. The method of claim 32, wherein said predetermined pacing interval is the VV-interval.

39. The method of claim 32, wherein said specified intervals are in the range of 6-24 hours.

40. The method of claim 32, wherein the pacing interval is varied in relation to said PIₒₚₜ during said predetermined PI search session.
